(19) United States
(12) Patent Application Publication
(10) Pub. No.: US 2017/0065249 A1
(43) Pub. Date: Mar. 9, 2017

(54) METHODS AND PROBES FOR VAGINAL TACTILE AND ULTRASOUND IMAGING

(71) Applicant: Vladimir Egorov, Princeton, NJ (US)

(72) Inventor: Vladimir Egorov, Princeton, NJ (US)

(73) Assignee: Advanced Tactile Imaging Inc., Trenton, NJ (US)

(21) Appl. No.: 15/249,672

(22) Filed: Aug. 29, 2016

Related U.S. Application Data
(60) Provisional application No. 62/215,227, filed on Sep. 8, 2015.

(51) Int. Cl. A61B 8/12; A61B 5/00; A61B 8/00; A61B 8/08; A61B 1/303

(52) U.S. Cl. CPC A61B 8/12; A61B 8/485; A61B 1/303; A61B 8/4416; A61B 8/5207; A61B 8/5223; A61B 8/5261; A61B 8/54; A61B 8/4254; A61B 5/4884; A61B 5/4337; A61B 8/08

(57) ABSTRACT

A vaginal probe is equipped with tactile sensors and ultrasound elements and configured for simultaneous acquisition of tactile images and ultrasound images for the same portion of vaginal tissues and pelvic floor muscles. The probe is configured for placement into vagina to record tactile images and ultrasound images in static, during tissue deformation as well as pelvic floor muscle contraction. Acquired and recorded tactile data are transmitted to a data processor for composing elasticity images of pelvic floor structures and muscle functional images and visually presenting thereof on a display.

### CROSS-REFERENCE TO RELATED APPLICATIONS

This patent application claims a priority date benefit from the U.S. Provisional Patent Application No. 62/215,227 filed Sep. 8, 2015 by the same inventor and with the same title, incorporated herein in its entirety by reference.

### GOVERNMENT-SUPPORTED RESEARCH

This invention was made with the US Government support under grant No. SB1AG034714 awarded by the National Institute on Aging. The Government has certain rights in this invention.

### FIELD OF THE INVENTION

The present invention generally relates to female pelvic floor imaging. Specifically, the invention describes methods and devices for providing vaginal tactile and ultrasound imaging to assess the conditions of the female pelvic floor.

### BACKGROUND

Pelvic organ prolapse (POP) is the abnormal descent or herniation of the pelvic organs from their normal attachment sites or their normal position in the pelvis. The conditions are often associated with concomitant pelvic floor disorders, including urinary and fecal incontinence, pelvic pain, sexual dysfunction, voiding dysfunction and social isolation. A recent study provided a projection that by the year 2050, 43.8 million women, or nearly 33% of the adult female population in the U.S., would be affected by at least one troublesome pelvic floor disorder. The lifetime risk of undergoing surgery for prolapse or urinary incontinence is near 20%.

Urinary incontinence (UI) is a storage symptom and defined as the complaint of any involuntary loss of urine. The most common type of UI is stress urinary incontinence (SUI), defined as the complaint of involuntary leakage on effort or exertion, or on sneezing or coughing. Estimates of prevalence vary depending on the affected population and definition of UI. Using the inclusive definition of any leakage at least once in the past year, the prevalence ranges from 25% to 51%.

Female pelvic floor comprise the pelvic diaphragm muscles (pubococcygeus, puborectalis, and iliococcygeus, together known as the levator ani), the urogenital diaphragm muscles (ischiocavernosus, bulbospongiosus, and transverses perinei superficialis, together known as the perineal muscles); and the urethral and anal sphincter muscles. These muscles interrelated to each other both anatomically and functionally. Normal action of the pelvic floor muscles (PFM) has been described as a squeeze around the pelvic openings and an inward lift. The pelvic floor disorders result from of neuro-urinary pathology as well as muscle functional impairment due to changes in biomechanical properties of soft tissues associate with age. The anatomy of the pelvic floor is complex and clinical examination alone is often insufficient to diagnose and assess pathology. A large number of these patients suffer for many years without proper treatment often due to the lack of objective findings necessary to plan proper treatment. Because abnormalities of the different pelvic compartments are frequently interrelated, their thorough diagnostic characterization is paramount for the proper disease management and treatment. That is why pelvic floor muscles characterization and diagnosis must include dynamic/functional stress-and-strain imaging to allow biomechanical (force) measurements. Real-time fusion of tactile (stress) and ultrasound (strain) imaging may deliver such possibility. The quantitative and objective imaging data for PFM conditions may allow an effective treatment and monitoring.

The treatment options for POP and SUI include surgery and pelvic muscle training. An invasive surgical approach is considered as the ultimate treatment for both POP and SUI. It was found that surgical treatments of recurrent SUI are associated with high failure rates. Physical training and medications are often not effective for treatment of UI. There is a need for the objective and quantitative pre- and post-surgery assessment of pelvic floor conditions to improve evidence-based management in urogynecologic surgeries. Determining the most effective and durable repair with lowest morbidity rate and cost is imperative.

Tactile Imaging is a medical imaging modality that translates the sense of touch into a digital image. The tactile image is a function of P(x,y,z), where P is the pressure on soft tissue surface under applied deformation and x,y,z are coordinates where pressure P was measured. Functional Tactile Imaging translates muscle activity into dynamic pressure pattern P(x,y,t) for an area of interest, where t is time and x,y are coordinates where pressure P was measured. Muscle activity may include a variety of types such as muscle voluntary contraction, involuntary reflex contraction, involuntary relaxation, specific maneuvers (e.g Valsalva maneuver) [van Raalte H. Egorov V. Tactile imaging markers to characterize female pelvic floor conditions. Open Journal of Obstetrics and Gynecology 2015; 5: 505-515, incorporated herein by reference in its entirety].

Tactile imaging solutions were developed for breast, prostate and vagina, as described in U.S. Pat. Nos. 6,620,115; 6,142,959; and 8,187,208, all incorporated herein by reference in their respective entireties. There is a need in imaging and accurate quantitative assessment of pelvic floor muscles and connective tissues for the effective management of the disorders. Therefore there is a need to provide methods and devices for acquiring vaginal and pelvic floor structure images by multiple imaging modalities which bring complementary information.

### SUMMARY

The object of the present invention is to overcome the drawbacks of the prior art and to provide novel methods and devices for objective characterization of the female pelvic floor conditions.

Another object of the invention is to provide methods and devices for objective characterization and real-time visualization of contractile capabilities of pelvic floor muscles.

A further object of the invention is to provide methods and devices for objective characterization and real-time visualization of a biomechanical condition of the connective ligaments and fasciae in the female pelvic floor.

A further yet object of the invention is to provide methods and devices for recording tactile images and ultrasound strain images for vaginal and pelvic floor tissues during vaginal wall deformations by a vaginal probe.

Another object of the invention is to provide methods and devices for objective visualization and real-time detection of damaged/weak pelvic floor muscles by recording a tactile response and ultrasound image changes during muscle contractions.

Another yet object of the invention is to provide methods and devices for composing tissue elasticity images from tactile images and ultrasound strain images.

Another object of the invention is to provide methods and devices for composing muscle functional images from tactile responses and ultrasound dynamic images.

Another yet object of the invention is to provide methods and devices for characterizing pelvic floor connective tissues and muscles as biomechanical elements based on tissue elasticity images and muscle functional images.

A further yet object of the invention is to provide methods and devices for objective diagnosis of a disease by comparing acquired tactile response and ultrasound images for a particular patient against respective normal values obtained from clinical data collected from a number of patients with known clinical status.

Disclosed in this description are various novel methods and probes for vaginal tactile and ultrasound imaging. Using a single probe with combined tactile and ultrasound imaging capabilities may improve technical capability, scientific knowledge and clinical practice in urogynecology and obstetrics and lower the actual costs of pelvic floor treatment.

In embodiments, a method for vaginal tactile and ultrasound imaging may include the steps of:
a. providing a vaginal probe equipped with a plurality of tactile sensors and a plurality of ultrasound elements positioned adjacent thereto,
b. inserting the vaginal probe into a vagina along a vaginal canal, and
c. simultaneously acquiring tactile images representing tissue stress and ultrasound images representing tissue strain for the same portion of vaginal and pelvic floor tissues during vaginal wall deformations.

Additional method steps may include applying tissue deformations to the vaginal walls while acquiring tactile and ultrasound images—via probe manipulations or muscle contractions.

Once tactile response data and ultrasound imaging data is acquired, additional steps of the method may include data processing to compose tissue elasticity images, muscle functional images as well as comprehensive tissue biomechanical characterization.

A probe for vaginal tactile and ultrasound imaging may include:
a probe housing with a front portion suitably shaped or tapered for atraumatic insertion into a vaginal canal to cause vaginal tissue deformation away from a center of said vaginal canal,
a plurality of tactile sensors forming together a tactile array located over at least a portion of the probe housing and configured to record a tactile image of vaginal walls when in contact therewith,
a plurality of ultrasound elements forming together an ultrasound array located adjacent to the plurality of tactile sensors over the same portion of the probe housing,
with the tactile array and the ultrasound array configured to record tactile images and ultrasound images for the same portion of vaginal and pelvic floor tissues, and
a controller operably connected to the probe housing comprising a data processor configured for acquiring these tactile images and ultrasound images.

In embodiments, the controller may be further configured and programmed to perform data processing steps outlined above. To objectively characterize tissue compression, a probe motion tracking system may also be provided. Such motion tracking system may include accelerometers and/or gyroscopes capable to detecting at least angular movements of the probe.

### BRIEF DESCRIPTION OF DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates vaginal probe location after its insertion during recording of a tactile response and acquisition of ultrasound images from two opposing vaginal walls;

FIG. 2 presents one embodiment of a probe for vaginal tactile and ultrasound imaging;

FIG. 3 presents a flow chart of tactile and ultrasound data acquisition for female pelvic floor;

FIG. 4 is a flow chart illustrating an embodiment of a method for vaginal tactile and ultrasound imaging.

FIG. 5 presents a block-diagram of a method for vaginal tactile and ultrasound imaging and guided female pelvic floor therapy.

### DETAILED DESCRIPTION OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Specific terms are used in the following description, which are defined as follows:
“tactile sensor” is the sensor capable to measure an applied force averaged per sensor area or pressure and transform it into an electrical signal to be used in tactile image formation;
“ultrasound element” is the sensor capable to emit and receive an acoustic wave and transform it into an electrical signal to be used in ultrasound image formation;
“stress” is a force per unit of area (pressure) measured at surface of a vaginal wall;
“strain” is a soft tissue displacement under tissue deformation;
“muscle function” is capability of muscle to contract; which is measured as pressure change on vaginal wall during muscle contraction;
“muscle strength” is capability of muscle to generate force measured as a force change on vaginal wall during muscle contraction.

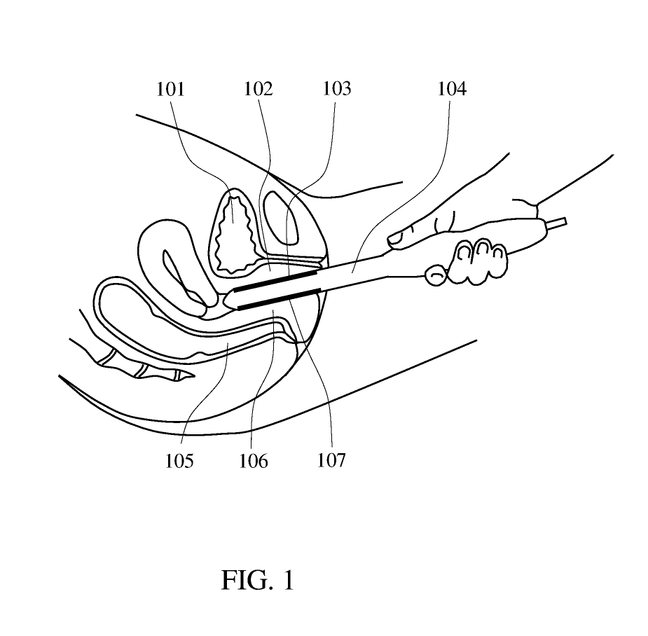

FIG. 1 illustrates position of a probe 104 after its insertion into vagina. Bladder 101 and rectum 105 are shown for anatomical references. The same probe position is used for recording of a tactile images and ultrasound images from one or both sides of vaginal canal. Recording of tactile images from two opposing vaginal walls contacting anterior 102 and posterior 106 compartments may be provided by means of two tactile sensor arrays consisting of a plurality of tactile sensors placed along the black lines 103 and 107 on both sides of the probe 104. Recording of ultrasound images may be provided by means of two ultrasound arrays consisting of a plurality of ultrasound elements placed along the same black lines 103 and 107 on both sides of the probe 104. The tactile and ultrasound arrays allow recording of respective signals during tissue deformation by the probe 104 and/or during pelvic floor muscle contraction as well as pelvic floor muscles at rest. Tactile measurements at muscle rest provide data for muscle tone, which is considered as important characteristic of muscle conditions. The pelvic floor muscle contraction may take a form of a voluntary muscle contraction, an involuntary muscle contraction, and muscle contraction during Valsalva maneuver.

To accurately record tactile and ultrasound images during muscle contraction, the probe 104 must be held in place without any displacements along vaginal canal, ideally by keeping the probe oriented in parallel to vaginal canal. The patient may be placed in a lithotomy position during the probe 104 insertion and imaging. Furthermore, the patient may be asked to contract vaginal muscles to enable recording of tactile and ultrasound signals on the flat rigid surface 103 and 107 of the probe 104. The patient may be also asked to follow specific instructions from a medical professional used in clinical practice for pelvic floor voluntary and involuntary (cough) muscle contraction as well as specific muscle maneuver (Valsalva).

The probe 104 may have at least one of rectangular, ellipsoidal or circular cross-sections. In embodiments, the probe 104 is shaped for atraumatic insertion into vagina and may have a generally rectangular cross-section with rounded edges and angles, so that smaller sides of the probe 104 may be equipped with at least some of the tactile sensors and ultrasound elements. A suitable lubricating gel may be used with the probe 104 insertion into vagina.

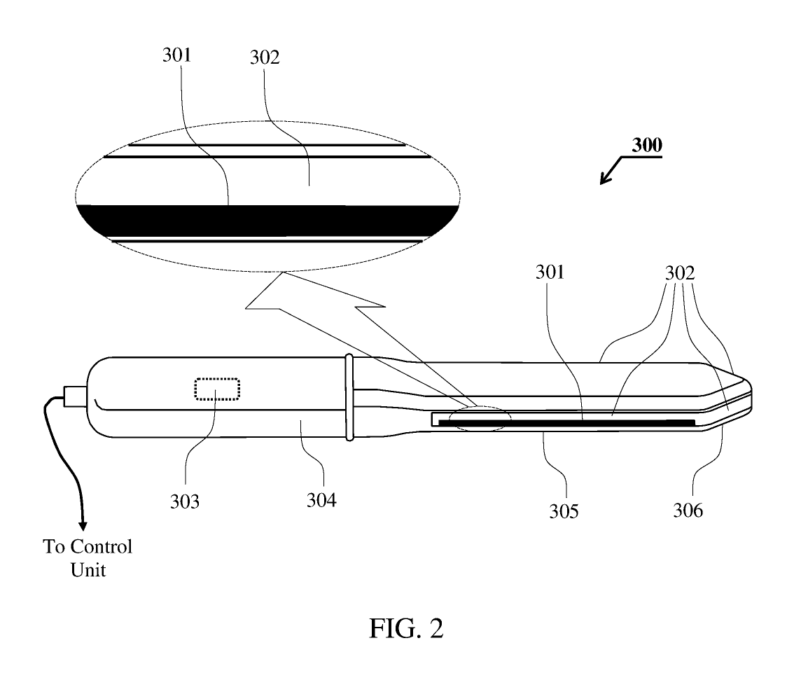

FIG. 2 presents an exemplary embodiment of a probe 300 for vaginal tactile and ultrasound imaging. Probe housing has a handle 304 and an elongated portion with two parallel opposite sides 305 and a front tapered portion 306 extending from the handle 304. The elongated portion of the probe housing may support a plurality of tactile sensors forming together a tactile array 302 located over at least a portion of the probe housing in a predefined relationship of tactile sensor positions to each other and to the probe housing. The tactile array 302 is configured to record a tactile response from vaginal walls.

The elongated portion of the probe housing may also house a plurality of ultrasound elements forming together an ultrasound array 301 located over at least a portion of the probe housing in a predefined relationship of element positions to each other and to the probe housing. The ultrasound array 301 is configured to acquire ultrasound images for soft tissues within a pelvic floor from two opposite sides of the vaginal canal.

Importantly, both the tactile array 302 sensors and the ultrasound array 301 elements may be positioned close or adjacent to each other so that both respective tactile image and ultrasound image may be acquired at the same time and for the same portion of the vaginal tissues.

The vaginal probe 300 may be operably connected to a control unit, which may be configured to provide a clinician with a control interface for operating the vaginal probe 300 as well as a suitable display for visualizing all signals acquired during the procedure.

Further referencing to FIG. 2, the tactile array 302 and ultrasound array 301 may be suitably configured to acquire their respective tactile and ultrasound signals from a portion or the entire vaginal canal. A certain minimum resolution may be required to be achieved by these imaging modalities. A minimum linear resolution of at least of 5 mm or better may be suitable for tactile sensors. For ultrasound sensors at least 2 mm resolution or better may be required. The tactile and ultrasound signal acquisition may be synchronized in time. The tactile sensors and ultrasound elements may have adjacent spatial locations along elongated portion of the probe.

The probe 300 may further be equipped with an orientation sensor 303, which allows measurement of probe angular deviation (elevation, rotation and azimuth). The orientation sensor 303 may serve as a motion tracking system and may include at least one of an accelerometer, a magnetometer, and a gyroscope.

Tactile signals from the tactile array 302 may further allow determination of insertion depth of the probe into a vagina. Both the orientation and probe insertion depth may be used as part of the function of a probe motion tracking system, which provides detection of probe position relative to pelvic floor organs.

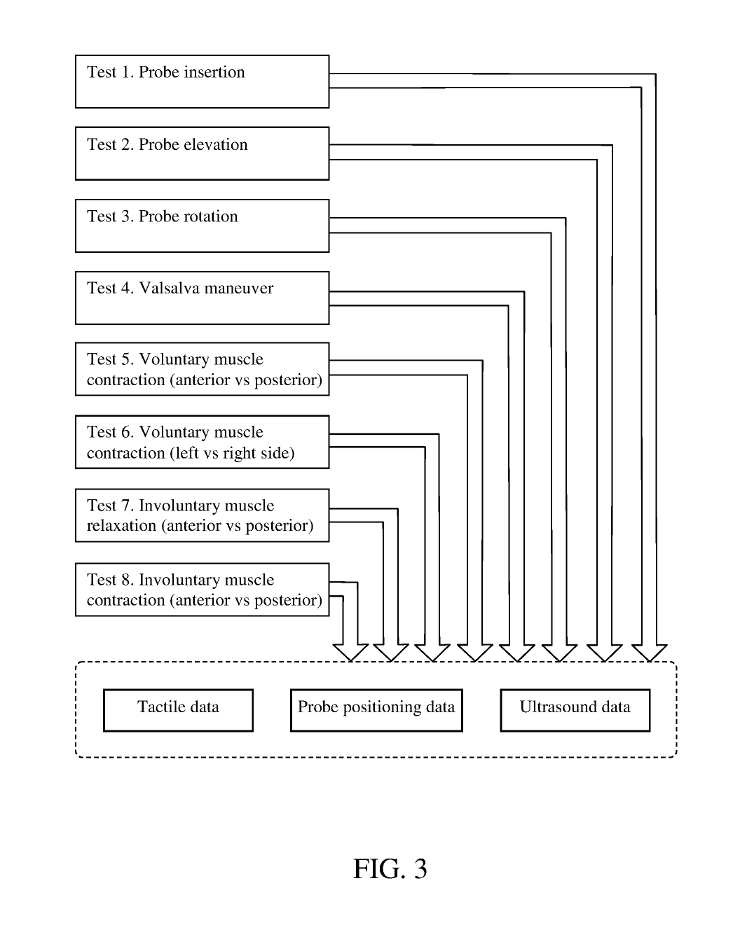

FIG. 3 presents an exemplary flow chart of tactile, ultrasound and probe positioning data acquisition during a patient examination with a vaginal probe of the invention. The pelvic floor examination procedure may include up to 8 tests as shown in FIG. 3:
Test 1: Probe insertion
Test 2: Probe elevation
Test 3: Probe rotation
Test 4: Valsalva maneuver
Test 5: Voluntary muscle contraction (anterior vs posterior)
Test 6: Voluntary muscle contraction (left vs right side)
Test 7: Involuntary muscle relaxation
Test 8: Involuntary muscle contraction

These tests may be used to acquire the following information:

Test 1: Tactile images and ultrasound images for vaginal anterior and posterior compartments along the entire vagina; tissue elasticity of vaginal surround (within 20-30 mm) may be calculated.

Test 2: Tactile images and ultrasound images for mid and apical anterior and posterior compartments, which relate to pelvic floor support structures may be assessed; their elasticity and strength under allied deformation may calculated.

Test 3: Circumferential tactile images and ultrasound images of vaginal canal, structures anatomical size may be calculated.

Test 4: Muscle pressure dynamic and muscle tissue displacement during Valsalva maneuver may be recorded for the opposite sides along the entire vagina (anterior vs posterior); rest muscle tone and dynamic components can be observed and calculated.

Test 5: Pressure response and muscle displacement within the pelvic floor for the opposite sides along the entire vagina (anterior vs posterior); static and contraction amplitudes can be observed and calculated; tissue contractile displacement can be observed and calculated.

Test 6: Pressure response and muscle displacement within the pelvic floor for the opposite sides along the entire vagina (left side vs right side); static and contraction amplitudes can be observed and calculated; tissue contractile displacement can be observed and calculated.

Test 7: Pressure dynamic for involuntary relaxation of pelvic floor muscles (weakening) recorded along the entire vagina (anterior vs posterior); relaxation graphs (slope) can be observed and documented.

Test 8: Pressure response and muscle displacement for involuntary contraction of pelvic floor muscles during patient's cough within the pelvic floor for the opposite sides along the entire vagina (anterior vs posterior); rest tone and contraction amplitudes can be observed and calculated; tissue contractile displacement can be observed and calculated.

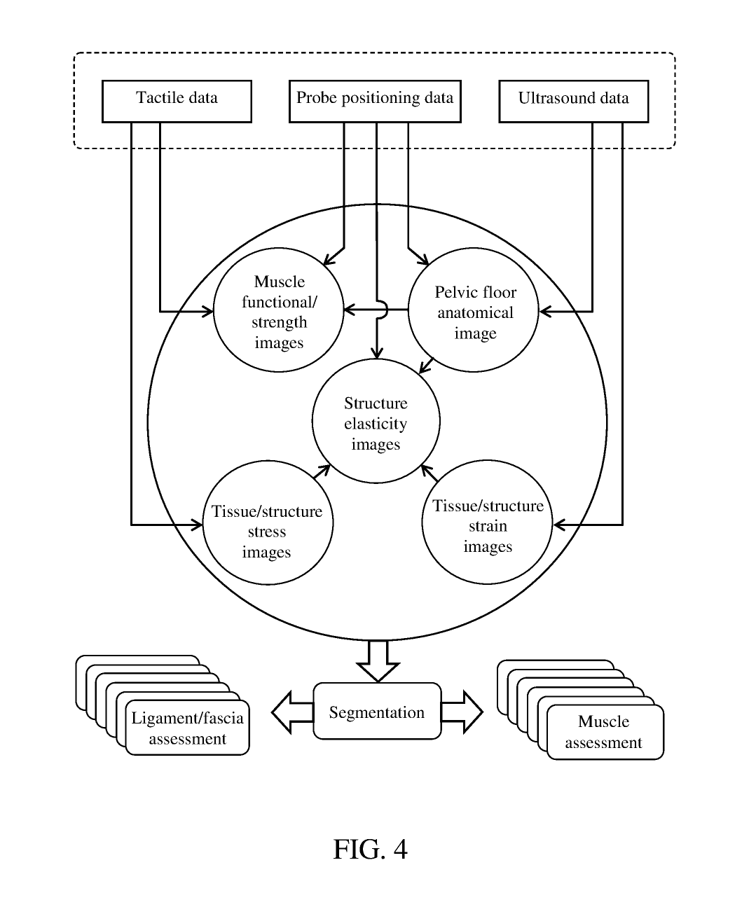

FIG. 4 is an exemplary flow chart illustrating a method for vaginal tactile and ultrasound imaging according to the present invention. Recorded tactile, ultrasound and probe positioning data during a patient examination with a vaginal probe allow composing:
a. pelvic floor anatomical image,
b. tissue/structure stress images,
c. tissue/structure strain images,
d. tissue/structure elasticity images, and
e. muscle functional/strength images.

The pelvic floor anatomical image may be generated by a conventional ultrasound imaging approach. The tissue/structure stress images may be derived from the tactile images under tissue deformation (see tactile imaging definition above). The tissue/structure strain images may be generated by following a set of pixels (with the use of motion tracking) within the ultrasound image for an area of interest. The tissue/structure elasticity images may be created as a ratio of stress to strain values for a set of pixels within superimposed (fused) tactile and ultrasound strain images for tissue deformation.

The muscle functional/strength images may be generated from ultrasound dynamic images and tactile data during pelvic floor muscle contraction. Further, all these five images may be segmented into ligamentous structures (pubourethral, arcus tendineus, cardinal, uterosacral, perineal) and muscles (puborectalis, pubococcygeus, pubovaginal, puboperineal, levator plate, ilicoccygeal). Further yet, all segmented structures may be quantitatively characterized based of digital data provided by elasticity images and muscle functional images.

Accurate probe motion tracking may allow for probe positioning data in real time within the image generated on the computer display. Imaging with moving probe is expected to increase the ultrasound as well as tactile image resolution.

Circumferential 3-D ultrasound and tactile image formation in test 3 (probe rotation) may be performed using probe orientation data from the motion tracking system.

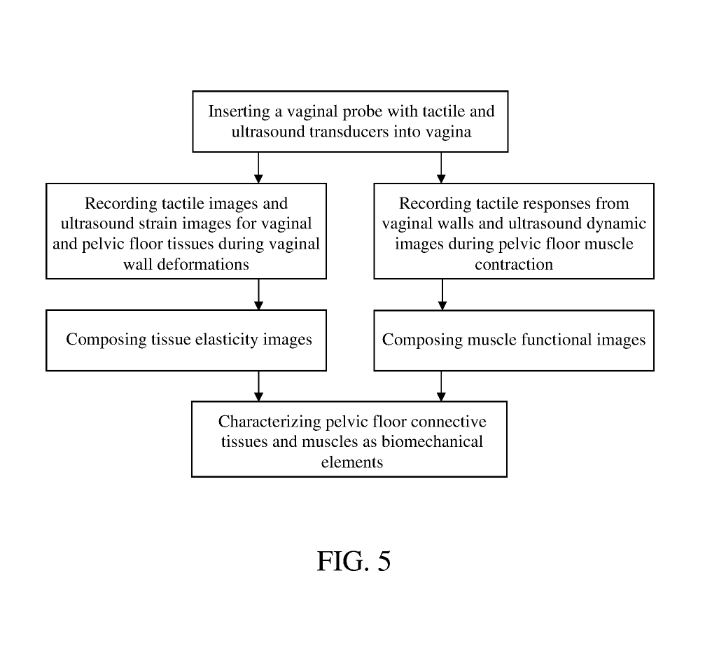

FIG. 5 presents an exemplary block-diagram of a method for vaginal tactile and ultrasound. A method for vaginal tactile and ultrasound imaging may include the steps of:
a. providing a vaginal probe equipped with a plurality of tactile sensors and a plurality of ultrasound elements positioned adjacent thereto,
b. inserting the vaginal probe into a vagina along a vaginal canal, and
c. simultaneously acquiring tactile images representing tissue stress and ultrasound images representing tissue strain for the same portion of vaginal and pelvic floor tissues during vaginal wall deformations.

Additional steps may include composing tissue elasticity images from the tactile images and the ultrasound images, composing muscle functional images from the tactile responses and the ultrasound dynamic images, and characterizing pelvic floor connective tissues and muscles as biomechanical elements based on the tissue elasticity images and the muscle functional images.

The vaginal wall deformations may be produced by vaginal probe insertion, by vaginal probe elevation and by vaginal probe rotation in the vaginal canal. In embodiments, tissue deformation may also be caused by pelvic floor muscle contractions generated by voluntary and involuntary reflex.

The acquisition of tactile and ultrasound data may be performed from one or two opposing vaginal walls along entire vaginal canal.

The step of composing tissue elasticity images may be conducted with the use of tissue stress data derived from the tactile images and tissue strain data derived from the ultrasound images.

The step of composing muscle functional images may be conducted with use of tactile response data overlaid onto contracting muscles identified in the ultrasound dynamic images.

The method may further include characterization and assessment of pelvic floor muscle, ligaments and fasciae.

The method may further include steps of biomechanical characterization of pelvic floor connective tissues and muscles by comparing tissue elasticity data and muscle function with a clinical database obtained from a group of patients with known diseased or healthy conditions so as to detect a presence or absence of diseased conditions using such comparison.

The method of the invention may further include assessment of pelvic floor muscle strength based on the tactile responses from vaginal walls.

In the method, the two opposing vaginal walls may be an anterior vaginal wall and a posterior vaginal wall. In the methods of the invention, two opposing vaginal walls may also be a left vaginal wall and a right vaginal wall.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method for vaginal tactile and ultrasound imaging, said method comprising the steps of:
a) providing a vaginal probe equipped with a plurality of tactile sensors and a plurality of ultrasound elements positioned adjacent thereto,
b) inserting said vaginal probe into a vagina along a vaginal canal, and
c) simultaneously acquiring tactile images representing tissue stress and ultrasound images representing tissue strain for the same portion of vaginal and pelvic floor tissues during vaginal wall deformations.

2. The method as in claim 1, wherein said step (c) is performed during pelvic floor muscle contractions.

3. The method as in claim 1, wherein said step (c) is performed while manipulating said vaginal probe inside the vaginal canal.

4. The method as in claim 1 further comprising a step (d) of composing an elasticity image using said tactile images and said ultrasound images.

5. The method as in claim 4, wherein said step (d) further comprising composing said tissue elasticity images using tissue stress data derived from said tactile images and tissue strain data derived from said ultrasound images.

6. The method as in claim 1 further comprising a step of presenting said tactile images and said ultrasound images either separately or overlaid together on a display.

7. The method as in claim 1 further comprising a step (e) of composing pelvic floor muscle functional images from said tactile images and said ultrasound images.

8. The method as in claim 7, wherein said step (e) further comprising composing said pelvic floor muscle functional images using said tactile images overlaid on contracting muscles identified in said ultrasound images.

9. The method as in claim 8 further comprising a step (f) of characterizing a pelvic floor based on said tissue elasticity images and said pelvic floor muscle functional images.

10. The method as in claim 9, wherein said step (f) further including characterizing pelvic floor ligaments and fasciae.

11. The method as in claim 9, wherein said step (f) further comprising characterizing pelvic floor connective tissues and muscles by comparing tissue elasticity data and muscle function against a clinical database obtained from a group of patients with known clinical status.

12. The method as in claim 4, wherein said step (d) further comprising an assessment of pelvic floor muscle strength based on said tactile images of vaginal walls.

13. A probe for vaginal tactile and ultrasound imaging, said probe comprising:
a probe housing with a front portion suitably shaped for atraumatic insertion into a vaginal canal to cause vaginal tissue deformation away from a center of said vaginal canal,
a plurality of tactile sensors forming together a tactile array located over at least a portion of said probe housing and configured to record a tactile image of vaginal walls when in contact therewith,
a plurality of ultrasound elements forming together an ultrasound array located adjacent to said plurality of tactile sensors over the same portion of said probe housing,
said tactile array and said ultrasound array are configured to record tactile images and ultrasound images for the same portion of vaginal and pelvic floor tissues, and
a controller operably connected to said probe housing comprising a data processor configured for acquiring said tactile images and said ultrasound images.

14. The probe as in claim 13, wherein said data processor is further configured for determining spatial deformation of at least a portion of said vaginal and pelvic floor tissues.

15. The probe as in claim 13, wherein said controller is further configured to compose a tissue elasticity image using said tactile images and said ultrasound images.

16. The probe as in claim 13, wherein said controller is further configured to compose pelvic floor muscle functional images from said tactile images and said ultrasound images.

17. The probe as in claim 13, wherein said probe housing comprises an elongated portion having parallel opposite sides and terminated with said front portion, said front portion is tapered, said plurality of tactile sensors are placed along said tapered front portion and along both opposite sides of said elongated portion of said probe housing.

18. The probe as in claim 17, wherein said plurality of ultrasound elements are placed along both opposite sides of said elongated portion of said probe housing.

19. The probe as in claim 17 further comprising a probe motion tracking system.

20. The probe as in claim 19, wherein said probe motion tracking system is configured for detecting angular deviation and probe insertion depth while within said vaginal canal.
